(19) United States
(12) Patent Application Publication
(10) Pub. No.: US 2023/0021437 A1
(43) Pub. Date: Jan. 26, 2023

(54) METHODS AND SYSTEMS FOR PATHOGEN MITIGATION IN ORGANIC MATERIALS

(71) Applicant: Plant Response Inc., Durham, NC (US)

(72) Inventors: Warren Clark Paris, Durham, NC (US); Rajib Biswas, Durham, NC (US)

(21) Appl. No.: 17/786,111

(22) PCT Filed: Dec. 16, 2020
(86) PCT No.: PCT/US2020/065395

§ 371 Date: Jun. 16, 2022

Related U.S. Application Data
(60) Provisional application No. 62/949,232, filed on Dec. 17, 2019.

(51) Int. Cl. A01N 63/32; C05F 11/08; C05F 5/00; A01P 1/00; A23K 10/18; A23K 30/00

(52) U.S. Cl. CPC A01N 63/32; A01P 1/00; A23K 10/18; A23K 30/00; C05F 5/00; C05F 11/08

(57) ABSTRACT

Methods and systems for inhibiting the proliferation of pathogenic microorganisms on organic biomass waste products without the need for pasteurization are described. The methods and systems allow conversion of organic waste into nutrient-rich fertilizers in a safe and efficient manner.

### CROSS-REFERENCE TO RELATED APPLICATION

This application claims priority to Provisional Application No. 62/949232, filed Dec. 17, 2019, the entire disclosure of which is hereby incorporated by reference herein for all purposes.

### BACKGROUND

Organic biomass is produced as waste products at all stages of agricultural production and food consumption. For example, in the food supply chain, organic biomass is produced from initial agricultural production stages to food processing, food distribution, retail sales, and final consumption stages. As a specific example, food scraps (i.e., remnant organic materials from the food supply chain that are not ultimately consumed) can originate from farms, grocery stores, food transportation companies, food processing companies, restaurants, and even from homes.

Considering a grocery store as an exemplary origin of organic biomass waste at one stage in the food supply chain, a significant amount of food scraps or waste is produced in the normal course of business when that is not saleable, past the expiration date, or is not aesthetically pleasing for display is discarded. The food scraps are consequently collected from the various departments of the grocery store and disposed of in a dumpster. This discarding of food represents a significant loss of energy and/or nutritive value. The loss scale of this inefficiency is amplified considering that such waste is similarly produced at earlier stages of production and preparation, and at later stages of incomplete consumption (e.g., at the home or restaurants).

In addition to energetic inefficiencies represented by the waste of food and other agricultural products, the disposal of such organic biomass waste products presents other problems and challenges.

Organic biomass waste products are susceptible to putrefaction. Putrefaction is the result of metabolic activity of microorganisms naturally found on the surface of the organic biomass, such as on vegetable food scraps, or microbial cross-contaminants from animal processing that colonize or reside on the surface of animal-based products. The rapid expansion of the microorganism populations manifests in the result of rapid, uncontrolled breakdown of the cellular structure and biochemical nutrients (e.g., vitamins, carbohydrates, lipids, proteins, etc.) that make up the biomass into simpler carbon molecules, ultimately producing acids, methane, hydrogen sulfide, and carbon dioxide. This decomposition of the biomass (e.g., food scraps) also results in foul-smelling organic compounds such as volatile fatty acids and foul-smelling polyamines and hydrogen sulfide. The metabolic activity occurring during putrefaction represents a major loss of thermodynamic energy and nutritive value, as well as a point where much of the utility of the biomass is irreparably lost.

In addition to the unpleasant smells associated with putrid biomass, the putrefaction by-products can also act as attracts for vermin (e.g., rodents) and insects, which can be vectors for disease. Moreover, cross-contamination present potentially dangerous proliferation of food-borne pathogens, such as E. coli, Salmonella and Listeria, which create unhealthy conditions and represent a risk of contamination to the food supply. Accordingly, commercial establishments that produce significant volumes of biomass, e.g., grocery stores, food production facilities, and restaurants) must have the food scraps hauled away at regular intervals, incurring significant and repeated costs.

Organic biomass, such as food scraps, is disposed of in a number of ways. For example, in the United States alone some 63 million tons of food scraps and waste are produced each year and nearly 58 million tons is committed to landfills for disposal. However, decomposing food waste is a nuisance and presents environmental issues, such as pollution hazards and issues, such as indicated above. Rainwater percolates through landfills, where food waste is deposited, and leads to leaching and, thus, contributing to the contamination of soils, surface water and ground water. Furthermore, putrid biomass waste emits greenhouse gases that subsequently cause significant environmental concern.

Attempts have been made to address certain environmental concerns of organic biomass disposal and to capitalize on the catabolic degradation process. One approach has been to conduct processing of the organic biomass using selected bacteria in an anaerobic environment to enhance the catabolic process. This process of anaerobic digestion attempts to capture the methane produced from the catabolic process and use the captured methane as an energy source. However, methane capture from organic biomass (e.g., food scraps recycling) has proven to be extremely inefficient and has, in some instances, been a net negative source of energy. Methane capture via anaerobic processing also still requires the grocery store or other location in the food supply chain to pay high disposal fees for removal and transport of the food scraps to the anaerobic digestion facility.

Another approach to dealing with the organic biomass disposal has been to compost the organic biomass. Composting is a controlled biological decay process that turns the organic biomass substrate into heat, carbon dioxide, ammonium, and incompletely decayed organic matter. The result of the controlled decay process is a humus-like material that is most often used as a soil amendment. However, the compost is characterized more by its value as a soil amendment resulting in greater moisture carrying capacity, than its intrinsic nutritive value. In addition, the nitrogen containing compounds produced by composting can be used to produce fertilizer. Significant amounts of the nutrients in the original organic biomass are still lost in the catabolic process resulting in the wasteful production of heat and carbon dioxide. This inefficiency can be further amplified by pasteurization efforts that are sometimes applied to eliminate pathogens from the final product. This heat, while effective at eliminating the pathogens has negative consequences on the nutritive quality of the fertilizer material because valuable vitamins, amino acids and other valuable nutrition is destroyed during this heating process. Ultimately, composting, like methane capture through anaerobic digestion, also still requires the grocery store or other location in the food supply chain to pay high disposal fees for removal and transport of the food scraps.

Many other systems and methods have been described for disposal of organic biomass waste (e.g., food scraps). These systems generally consist of methods for decreasing bulk volume of the waste and a) use of the shredded food waste as animal feed or b) disposal through the sanitary sewer system where the organic material is again catabolized (controlled or uncontrolled) by microorganisms from many different Domains and Phyla. Disposal in this manner results in much of the carbon and nitrogen material being lost through carbon dioxide or methane. Disposal of organics through the sanitary sewer system simply transfers the hazards and problems of decaying food waste to the local or regional water treatment plant, but still ultimately results in the loss of thermodynamic energy in the food scraps and the generation of greenhouse gases. Thus, previous attempts at addressing the nuisance of food scraps have sought value in the transport and disposal in landfills (so-called tipping fees) or in catabolic (degradative) byproducts of the decomposed food scraps such as methane capture.

Accordingly, a need remains for effective and inexpensive methods to inhibit the proliferation of pathogenic microorganisms on organic biomass waste products without the need for pasteurization so as to provide a safe and nutrient-rich product. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of inhibiting pathogenic microbial growth in biomass. The method comprises:
contacting the biomass with an effective amount of live non-pathogenic yeast;
agitating the biomass to distribute the yeast within the biomass to provide a yeast-stabilized biomass slurry; and
maintaining aerobic conditions in the slurry to permit yeast to grow aerobically.

In another aspect, the disclosure provides a method of inhibiting putrefaction in biomass. The method comprises:
processing a biomass to produce a substantially homogenized liquid slurry;
contacting the substantially homogenized liquid slurry with an effective amount of live non-pathogenic yeast;
agitating the substantially homogenized liquid slurry continuously to distribute the yeast within the substantially homogenized liquid slurry in aerobic conditions to provide a yeast stabilized biomass slurry;
filtering the yeast stabilized biomass slurry to remove macroparticles to produce a yeast stabilized biomass slurry filtrate; and
aerating the yeast stabilized biomass slurry filtrate.

In either aspect, the method can further comprise imposing one or more additional hurdle conditions to the yeast-stabilized biomass slurry and/or yeast stabilized biomass slurry filtrate.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 schematically illustrates an exemplary embodiment where the disclosed method of preventing or inhibiting pathogenic microbial growth (illustrated as “biopreservation”) is incorporated into a process to produce a refined biomass product from the initial organic biomass materials (e.g., food scraps). In this figure, F, pH, EC, a_{w}, Eh, and Pr indicate approximate points where in the process certain stresses (hurdles) are imposed on pathogens; “F” stands for increased temperature and pressure, and “PR” stands for biopreservation.

FIGS. 2A-2C are photographs of plates showing growth of E. coli and Salmonella spp. (combined) that were plated after co-incubation with S. cerevisiae for 0 minutes (FIG. 2A), 30 minutes (FIG. 2B), and 60 minutes (FIG. 2C). Each figure shows three plates corresponding to (left to right) growth on XLD plates, YPD plates, or saline/slurry control (i.e., no co-incubation with S. cerevisiae) on XLD plates. The assays are described in more detail in Example 4.

FIGS. 3A-3C are photographs of plates showing growth of E. coli and Salmonella spp. (combined) that were plated after co-incubation with S. cerevisiae, C. utilis, and C lipolytica (combined) for 0 minutes (FIG. 3A), 30 minutes (FIG. 3B), and 60 minutes (FIG. 3C). Each figure shows three plates corresponding to (left to right) growth on XLD plates, YPD plates, or saline/slurry control (i.e., no co-incubation with S. cerevisiae) on XLD plates. The assays are described in more detail in Example 4.

### DETAILED DESCRIPTION

The disclosure provides methods to inhibit the growth and proliferation of pathogenic microorganisms in organic biomass waste products to provide for a controlled catabolism process that does not require pasteurization. The method can be applied to efficiently produce an organic product that maintains a highly nutritive value and yet is safe for various uses, such as for fertilizer or animal feed. As described in more detail below, the inventors have established that co-incubation of pathogenic microorganisms with various yeast species in an organic slurry or slurry derived from food scraps sources results in the rapid reduction and often complete removal of the pathogenic microorganisms.

Without being limited to any particular theory, it is believed that the yeast not only competes with the microorganisms for nutritive resources in the organic substrate, but also creates conditions that are inhibitory to the growth and proliferation of the pathogenic microorganisms. Accordingly, the application of yeast provides a “hurdle” to the growth and survival of the microorganisms. This yeast-driven hurdle can be leveraged as part of a broader hurdle strategy to prevent proliferation of pathogenic yeast and even putrefaction of organic biomass waste products. “Hurdle” strategies, also known as “combination preservation” are conventionally known multi-pronged strategies to maintain microorganism stability or even prevent microorganism growth in substrates that might otherwise promote a proliferation of microorganism growth (e.g., food products.) The strategies can be specifically applied to prolong shelf-life of food and other products susceptible to putrefaction. Conventional hurdle approaches provide multiple challenges to microorganism growth by imposing suboptimal growth conditions such as restricted pH, temperature, pressure, moisture (water activity), salt content, electrical conductivity, and redox potential. Whereas any one of the restricted conditions alone might be somewhat detrimental to the microorganism in the substrate, the application of multiple factors combine synergistically to overcome the microorganism’s ability to thrive or even survive. This, in combination, the intensity of any individual hurdle may be set below the individual threshold to inhibit a target microorganism. While some microorganisms might be able to overcome one or a few hurdles individually, they are unable to overcome all hurdles in combination. Hurdle technologies and their application in the area of food preservation have been described, e.g., Tanaka, J. FoodProtect., vol. 49, no. 7, pp. 526-531 (July 1986), the contents of which are incorporated herein by reference.

The present disclosure presents a new hurdle that can be employed alone or in strategic combination with other hurdles such as modification of pH, temperature, pressure, water activity, electrical conductivity, and/or redox potential to achieve inhibition of pathogenic microorganism growth in organic biomass substrates such as agricultural and food biomass scraps products.

In accordance with the foregoing, the disclosure provides a method of inhibiting pathogenic microbial growth in biomass. The method comprises:
contacting the biomass with an effective amount of live non-pathogenic yeast;
agitating the biomass to distribute the yeast within the biomass to provide a yeast-stabilized biomass slurry; and
maintaining aerobic conditions in the slurry to permit yeast to grow aerobically.

The biomass can comprise food, food scraps, waste products, agricultural waste products, domestic yard waste products, and combinations thereof. In some embodiments, biomass can be an organic biomass that includes food scraps. Food scraps are remnant organic materials from the food supply chain that are not ultimately consumed. In some embodiments, food scraps refers to food components that have been deemed unsalable for any reason. In some embodiments, the food scraps have been served to customers but not eaten. In some embodiments, the biomass can be organic biomass that includes plant parts, such as grown and produced in yard maintenance or from agricultural production. The biomass can be solid (or a mix of multiple solid components), liquid, or a mixture of solid and liquid components.

In some embodiments, the method further comprises processing the biomass to produce a substantially homogenized liquid slurry prior to contacting with the effective amount of live non-pathogenic yeast. The term “substantially homogenized liquid slurry” encompasses liquids that possess solid chunks, particles, or incompletely liquefied fragments of organic biomass mixed therein. In some embodiments, the processing step comprises wetting the biomass with water. In some embodiments, the water is heated to a temperature from about 90° F. to about 130° F., such as 90° F., 100° F., 110° F., 120° F., 130° F., plus or minus 5° F. In other embodiments, the substantially homogenized liquid slurry is heated at least temporarily to about a temperature from about 90° F. to about 150° F., such as 90° F., 100° F., 110° F., 120° F., 130° F., 140° F., 150° F., or within 5° F. of any of the indicated temperatures. The processing step can also include steps of crushing or grinding the biomass to provide the substantially homogenized liquid slurry. In some embodiments, the remaining solid biomass component of the substantially homogenized liquid slurry has at least 75% of particles having a diameter less than 5 mm, less than 5 mm, or less than 1 mm.

The live non-pathogenic yeast comprises yeast, which can be any non-pathogenic yeast species that can grow under aerobic conditions. The yeast can function to release nutrients from biomass inputs and growth medium, and simultaneously outcompete and restrict growth of pathogenic microbes potentially present in the biomass. In some instances, the yeast contributes to environmental conditions that serve as a barrier or “hurdle” to pathogenic microbial maintenance and growth. In some embodiments, the live non-pathogenic yeast comprises yeast selected from the genera Saccharomyces or Candida, or combinations thereof. In some embodiments, the live non-pathogenic yeast comprises Saccharomyces cerevisiae, Candida utilis, or Candida lipolytica, or combinations thereof.

The live non-pathogenic yeast contacted with the biomass can be in any dosing form. In some embodiments, the yeast contacted with the biomass are dormant. In some embodiments, the yeast contacted with the biomass are dry, active yeast. In some embodiments, the yeast contacted with the biomass are metabolically active, e.g., actively growing and reproducing. For example, in some embodiments, the yeast contacted with the biomass are in a liquid inoculum. To illustrate, an exemplary liquid inoculum comprising biologically active yeast or combinations of yeast can be prepared in the following manner:
a. Small batches of cultured yeast are increased through a series of 10-fold increases in growth media using additions of a macro supplement, sugar, homogenized nonpathogenic yeast (e.g., S. cerevisiae) and water.
b. Each multiplication is incubated for 24-48 hours at 15-30° C. with aeration.
c. The end result of this process is liquid inoculum. For example, an inoculum batch can be comprised of:
   i. about 92.0 ± 5% water
   ii. about 1.5 ± 0.5% homogenized non-pathogenic yeast (e.g. S. cerevisiae)
   iii. about 2.0 ± 0.5% sugar
   iv. about 4.5 ± 1% macro supplement
d. The inoculum is then added to the biomass as described herein

The amount of yeast live non-pathogenic yeast contacted with the biomass can be determined based on several factors, including the amount, content, and condition of the particular biomass. As used herein, the phrase “effective amount” refers to a sufficient amount of live non-pathogenic yeast such that the pathogenic microbial growth is measurably inhibited as compared the same or similar biomass where the live non-pathogenic yeast is not added. The presence of or growth of pathogenic microorganisms can be readily determined by, e.g., culture assays, assaying of toxins produced by pathogenic microorganisms, or assaying products of pathogenic microorganism catabolic activity. In some embodiments, the presence or growth of the pathogenic microorganisms can be inferred by measuring putrefaction, including measuring volatile fatty acids and foul-smelling polyamines and hydrogen sulfide. In some embodiments, the effective amount of live non-pathogenic yeast is at least 1E³ CFU/mL, at least 5E³ CFU/mL, at least 1E⁴ CFU/mL, at least 5E⁴ CFU/mL, at least 1E⁵ CFU/mL, at least 5E⁵ CFU/mL, or at least 1E⁶ CFU/mL.

The effective amount of live non-pathogenic yeast is added to the biomass continuously while agitating the biomass to create the yeast-stabilized biomass slurry. The agitating not only distributes and disperses the yeast throughout the biomass, but also promotes aerobic conditions throughout the biomass. The addition can be in a single dose, multiple discrete doses, or continuous addition over a period of time. In some embodiments, only an initial amount of yeast is added to establish a population that can grow. In other embodiments, the initial introduction of the live non-pathogenic yeast is supplemented by additional steps of adding live non-pathogenic yeast to either maintain a constant population in the biomass or increase the population in the biomass. Additional administrations of live non-pathogenic yeast can be determined based on various key performance indicators (KPIs) of the biomass, including pH, select bacterial/pathogen concentrations, seed organism (i.e., live-nonpathogenic yeast) concentrations, or combinations there. In some embodiments, the live non-pathogenic yeast are contacted in one dose or in multiple discrete doses over time sufficient to maintain a population of live of at least 1E³ CFU/mL, at least 5E³ CFU/mL, at least 1E⁴ CFU/mL, at least 5E⁴ CFU/mL, at least 1E⁵ CFU/mL, at least 5E⁵ CFU/mL, or at least 1E⁶ CFU/mL. In some embodiments, a concentration of live non-pathogenic yeast at or less than about 1E⁴ CFU/mL, signals a need to add additional live non-pathogenic yeast. In some embodiments, the additional live non-pathogenic yeast are added until the concentration is about or exceeds 1E⁴ CFU/mL. In some embodiments, the live non-pathogenic yeast are contacted in one dose or in multiple doses over time sufficient to maintain a bacterial/pathogen concentration less than 1E⁴ CFU/mL, such as less than 5E³ CFU/mL or less than 1E³ CFU/mL. In some embodiments, a concentration of bacterial/pathogen concentration at or greater than 1E⁴ CFU/mL, 5E³ CFU/mL, or 1E³ CFU/mL indicates a need to add additional live non-pathogenic yeast.

In some embodiments, the method further comprises adding a micro-nutrient comprising yeast lysate residue to the biomass. Typically, the micro-nutrient supplement is added after the biomass has been contacted with the yeast and converted to the yeast stabilized slurry but it can also be added prior to the contacting with the effective amount of live non-pathogenic yeast. The micro-supplement provides micronutrients and growth factors that promote maintenance and growth of the yeast in the biomass. An exemplary micro-supplement can comprise non-pathogenic yeast or components thereof (e.g., S. cerevisiae (obtainable from, e.g., breweries), and/or yeast cell walls (e.g., from Hangzhou Focus Corp, Hangzhou, CN)). In some embodiments, the non-pathogenic yeast or components thereof (e.g., S. cerevisiae) undergoes processing by mechanical filtration to remove large particles and homogenization. After homogenization, the yeast is optionally filtered again. The final micro-supplement can be about 10% solids and 90% water by weight. Yeast lysate residue (referred to under the trade name as “yeast cell walls”) comprises the solids separated from the mother liquor of a yeast slurry after a heat-induced autolysis step. Commercial yeast cell walls is typically delivered as a dry powder, it can be substituted for prepared S. cerevisiae in a 1:10 ratio, with the balance of the mass made up of water or additional prepared homogenized liquid biomass slurry.

In some embodiments, the method further comprises adding a macronutrient supplement to the yeast-stabilized biomass slurry. The macronutrient supplement provides additional nutrients to the biomass that serve as sources of, e.g., nitrogen, phosphorus, potassium, sulfur and/or carbon to promote yeast growth. Macronutrient supplement ingredients can also provide all, some or a significant proportion of micronutrients including organic acids, vitamins and minerals. In some embodiments, the macronutrient is at least partly or completely derived from plants. To promote nutrient availability from the macronutrient supplement, the supplement can optionally be treated first with enzymes. Once treated, macronutrient supplement ingredients can be mixed and added to the biomass in quantities sufficient to produce the desired nutrient content. As with the micronutrient supplement, the macronutrient supplement is typically added after the biomass has been contacted with the yeast and converted to the yeast stabilized slurry. However, the macronutrient supplement can also be added prior to the contacting with the effective amount of live non-pathogenic yeast.

In some embodiments, maintaining aerobic conditions comprises agitating the yeast-stabilized biomass slurry continuously or periodically. The slurry can be simultaneously ventilated with gas comprising oxygen. In other embodiments, gas comprising oxygen (e.g., air) can be infused or aerated into or over the slurry, such as from a compressed air source.

The reduction of pathogenic microbial growth can be expressed as a comparison to pathogenic microbial growth in equivalent biomass that is not contacted with the live nonpathogenic yeast. The pathogenic microbes can be any microbe (e.g., bacteria) that promotes putrefaction or can otherwise simply grow in the biomass. In some embodiments, the pathogenic microbes are known human pathogens, such as food-borne pathogens. For example, in some exemplary and non-limiting embodiments, the pathogenic microbes are selected from the genera Lactobacillus, Enterobacter, Salmonella, and Escherichia.

The disclosed method can also incorporate application of various other hurdles conditions (i.e., detrimental environmental conditions) to further control or inhibit the growth of pathogenic microorganisms in the biomass. As indicated above, any one hurdle may not necessarily impose a lethal condition on a target microorganism and could even facilitate selection for pathogen organisms able to resist the single hurdle. However, due to the synergistic effects of multiple hurdles, the intensity of individual hurdles may be applied at below a threshold required for microbial inhibition and could avoid development of pathogen resistance. While some microorganisms might be able to overcome one or a few hurdles individually, they are unable to overcome all hurdles in combination (e.g., in simultaneous and/or sequential combination). The introduction of the live nonpathogenic yeast to the biomass, as described above, provides an important hurdle to the growth of pathogenic microorganisms, which can be combined with one or more additional hurdles, as described below, to further enhance the anti-microbial environment in the biomass. This can prevent growth of undesired microbial growth, e.g., growth of pathogenic microorganisms, and can ultimately reduce, prevent, or slow putrefaction.

The one or more additional hurdles, as described below, can each be individually applied concurrently with or independently from the introduction of the live-nonpathogenic yeast to the biomass, as described above. The application of the one or more additional hurdles can be for similar durations or different durations with respect to each other and with respect to the introduction of the live-nonpathogenic yeast to the biomass. Any combination of the additional hurdles can be applied. In some embodiments, one or more of the additional hurdles are applied for a period that is concurrent or at least overlaps with the introduction of the live-nonpathogenic yeast to the biomass. In some embodiment, one or more of the additional hurdles are applied at a time after the introduction of the live nonpathogenic yeast to the biomass is complete. In further embodiments, additional live nonpathogenic yeast are introduced to the biomass in a second or subsequent dose that overlaps with the application of the one or more additional hurdles. It should be appreciated that the different hurdles need not be applied or introduced to the biomass mixture at the same location. For example, the present disclosure encompasses embodiments where the live nonpathogenic yeast are introduced to the biomass in a first tank at a first location (e.g., such as the source of the biomass, such as at a grocery store that produces food scraps). While one or more additional hurdles can be optionally applied in the first tank at the first location, the yeast-stabilized biomass slurry can be removed to a second location such as a production facility where additional one or more hurdles are applied.

The one or more additional hurdles are now discussed individually.

Thermal processing is a broad-spectrum pathogen reduction technique. However, excessively high temperatures, such as those used in pasteurization, can lead to reduction or loss of nutritive quality of the biomass substrate. Thus, moderately elevated temperatures can be applied. While such moderately elevated temperatures can still permit the growth of many microorganisms, fluctuations in temperature throughout the production process causes metabolic stress as organisms expend energy to adapt to the changing environment. The expendature of energy leads to metabolic exhaustion alone and/or in conjunction with other hurdles, resulting in the death of the pathogenic microorganisms. In some embodiments, the method further comprises maintaining a temperature in the yeast-stabilized biomass slurry selected from about 50° F. to about 120° F. The temperature can be maintained for at least about 30 minutes and up to a timescale of days. Exemplary times include at least 30 minutes, 1 hour, 2 hours, 3 hours, 4 hours, 5 hours, 6 hours, 7 hours, 8 hours, 9 hours, 10 hours, 12 hours, 18 hours, 24 hours, 2.5 days, 3 days, 4 days or more. In some embodiments, the temperature is elevated to at least 70° F., at least 80° F., at least 90° F., at least 100° F., or at least 110° F. Any of these temperatures can be maintained for at least 30 minutes as described above.

In some embodiments, the method also comprises elevating the pressure imposed on the yeast-stabilized biomass. In many practical applications, the agitating and homogenization of the biomass, including in the resultant processed slurry forms, is performed mechanically. The mechanical agitation often imposes elevated pressure to at least a component of the biomass at a given time. By virtue of the biomass substrate circulating in the container during processing or agitating, eventually most or all of the biomass is subjected to elevated pressure for a duration of the method. However, the particular portion that experience elevated pressure can be constantly changing due to the agitating process. Thus, the elevated pressure can be imposed on at least a component of the biomass at any time point. In some embodiment, the elevated pressure is a pressure between about 2 bars and 18 bars, such as 2 bars, 3 bars, 4 bars, 5 bars, 6 bars, 7 bars, 8 bars, 9 bars, 10 bars, 11 bars, 12 bars, 13 bars, 14 bars, 15 bars, 16 bars. This elevated pressure is applied to at least a portion (and in some embodiments all) of the yeast-stabilized biomass slurry for a total of about a half hour over the course of homogenization/treatment. If the elevated pressure is a result of the particular agitation process, it will be applied as long as the slurry is agitated. Any given component of the slurry batch will receive about 30-120 seconds total time of elevated pressure after which a different component is cycled through the area of elevated pressure. Because the total processing time can be e.g., over 12 hours, the total cumulative time with application of elevated pressure in the batch can for at least about 30 minutes and up to a timescale of days. Exemplary times include at least 30 minutes, 1 hour, 2 hours, 3 hours, 4 hours, 5 hours, 6 hours, 7 hours, 8 hours, 9 hours, 10 hours, 12 hours, 18 hours, 24 hours, 2.5 days, 3 days, 4 days or more. In some embodiments, the pressure is maintained at a pressure selected from 5 bars to 16 bars for at least 30-120, e.g., about 60, seconds for any particular component of the batch slurry over the course of treatment.

As an example, deployment of a homogenizer with a maximum flow rate of 7000 L/hr provides a maximum process pressure of about 16 bars. This high-pressure homogenization can effectively inactivate many bacteria. Thus, while some microbes may be able to withstand this hurdle of enhanced pressure, the number of microbes is reduced and the remaining bacteria can suffer metabolic stress as a result.

Relative acidity (i.e., lower pH) can serve as an additional hurdle that can impose pathogen reduction and preservation of biomass products. A lowered pH of the environment further increases the antimicrobial properties of certain weak organic acids by enhancing their ability to penetrate microbial cells and disrupt normal metabolic processes. Thus, in some embodiments, the method further comprises maintaining the yeast-stabilized biomass slurry at a pH less than 5 for at least 30 minutes. In further embodiments, the yeast-stabilized biomass slurry is maintained at a pH of 4.2±0.5 for at least 30 minutes. Exemplary times for maintaining a lowered pH include at least 30 minutes, 1 hour, 2 hours, 3 hours, 4 hours, 5 hours, 6 hours, 7 hours, 8 hours, 9 hours, 10 hours, 12 hours, 18 hours, 24 hours, 2.5 days, 3 days, 4 days or more. The step of maintaining the pH can comprise adding one or more acids to the yeast-stabilized biomass. Exemplary non-limiting acids for this purpose include lactic acid, citric acid, succinic acid, and volatile fatty acids. Additionally, the acids can be part of or result from the addition of various macronutrients or other additives encompassed herein. While conditions of lowered pH may not completely eliminate all target pathogenic microorganisms, the surviving microorganisms will likely be metabolically stressed and more susceptible to other detrimental factors, such as imposition of other hurdle factors.

Water activity often has a significant influence whether the growth of an organism will be reduced in a biomass product. Water activity can be combined with other hurdle factors such as temperature, pH, and redox potential to establish conditions that are inhibitory to pathogenic microorganisms. In some embodiments, the method further comprises maintaining the yeast-stabilized biomass slurry at a water activity less than 0.97 A_{w} for at least 30 minutes. As with the other hurdles described above, the water activity level can be imposed for at least about 30 minutes and up to a timescale of days. Exemplary times include at least 30 minutes, 1 hour, 2 hours, 3 hours, 4 hours, 5 hours, 6 hours, 7 hours, 8 hours, 9 hours, 10 hours, 12 hours, 18 hours, 24 hours, 2.5 days, 3 days, 4 days or more. In some embodiments, the water activity can be maintained at less than 0.95 A_{w}, 90 A_{w}, or 85 A_{w} for at least 30 minutes. Typically, when applied as a singular additional hurdle to pathogenic microbial growth, the water activity of about 0.85 A_{w} or below can be applied. However, when combined with additional hurdle factors, such as lowered pH, the water activity can be applied at a lower intensity, such as between (and including) about 0.95 A_{w} to about 85 A_{w} for at least 30 minutes.

In some embodiments, the method further comprises maintaining the yeast-stabilized biomass slurry at an electrical conductivity (EC) of 20.0±5 mS/cm for at least 30 minutes. Microbial susceptibility to electrical conductivity is due in large part to the high concentration of salts and dipolar molecules that lead to an inhibition of microbial growth. As with the other hurdles described above, the EC level can be imposed for at least about 30 minutes and up to a timescale of days. Exemplary times include at least 30 minutes, 1 hour, 2 hours, 3 hours, 4 hours, 5 hours, 6 hours, 7 hours, 8 hours, 9 hours, 10 hours, 12 hours, 18 hours, 24 hours, 2.5 days, 3 days, 4 days or more.

The oxidation-reduction or redox potential (Eh) is a measurement of a compound's ability to be oxidized and reduced. The redox potential (Eh) is measured in terms of millivolts (mV). During oxidation, electrons are transferred from an electron donor to an acceptor, which is reduced. Generally, the range at which different microorganisms can grow are as follows: aerobes +500 to +300 mV; facultative anaerobes +300 to -100 mV; and anaerobes +100 to less than -250 mV. The relationship of Eh to microbial growth in media is significantly affected by the pH, presence of salts and other constituents in the processed materials. In general, aerobic organisms need an environment that has a relatively high capacity to accept electrons (positive Eh), while anaerobes need an environment rich in electron donors (negative Eh). In our processing environment, the low Eh is unfavorable to aerobic organisms while strict anaerobes are exhausted by continuous mixing and aeration to maintain aerobic conditions throughout processing. Additionally, Eh can accentuate metabolic stress generated by pH and EC levels unfavorable for pathogenic growth. Thus, in some embodiments, the method further comprises maintaining the yeast-stabilized biomass slurry at a redox potential (Eh) selected from 0 mV to -200 mV for at least 30 minutes. As with the other hurdles described above, the Eh level can be imposed for at least about 30 minutes and up to a timescale of days. Exemplary times include at least 30 minutes, 1 hour, 2 hours, 3 hours, 4 hours, 5 hours, 6 hours, 7 hours, 8 hours, 9 hours, 10 hours, 12 hours, 18 hours, 24 hours, 2.5 days, 3 days, 4 days or more.

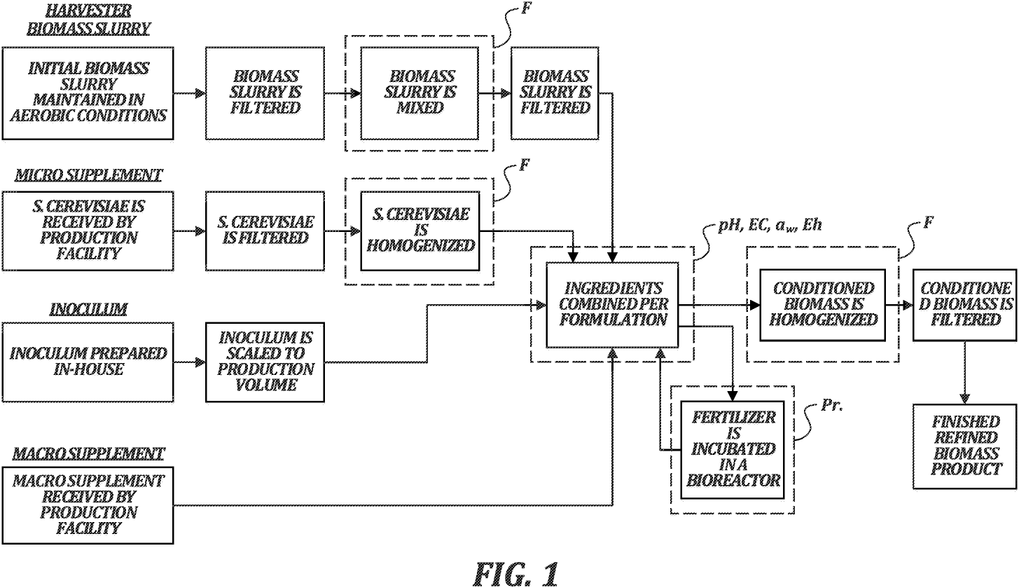

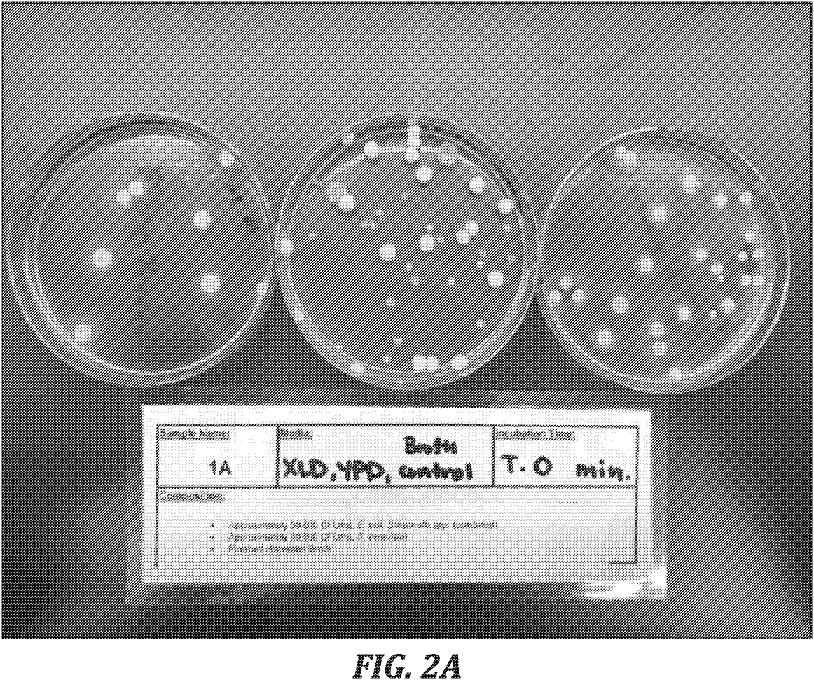

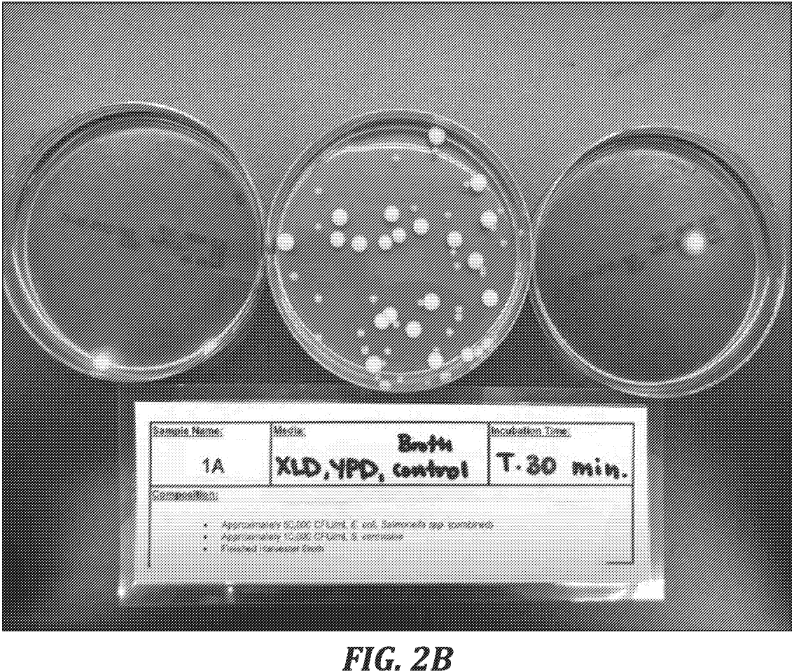

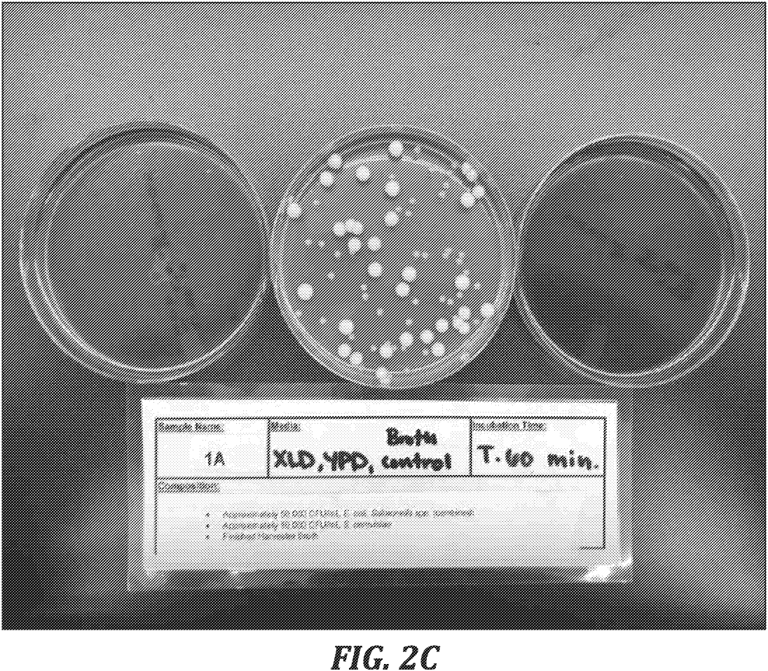

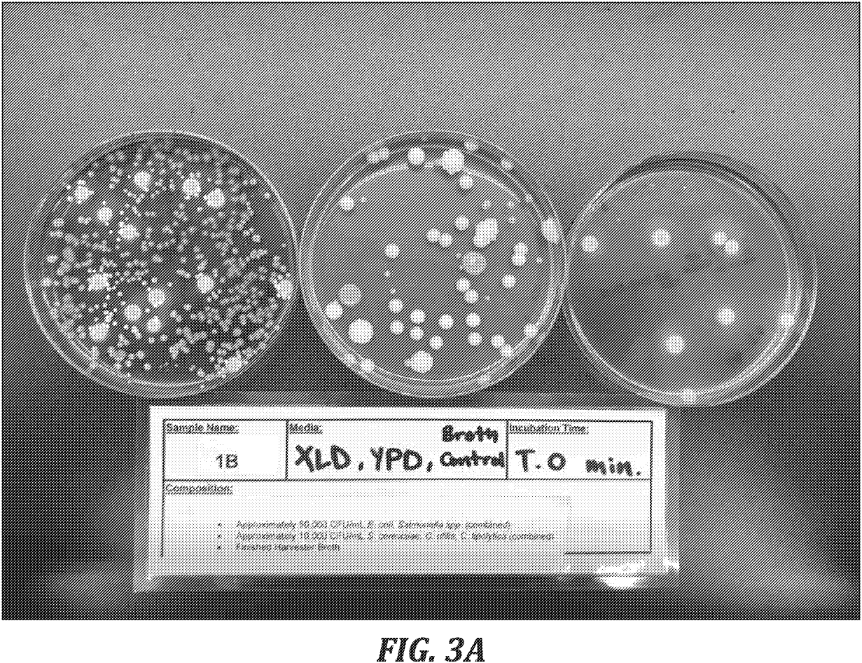

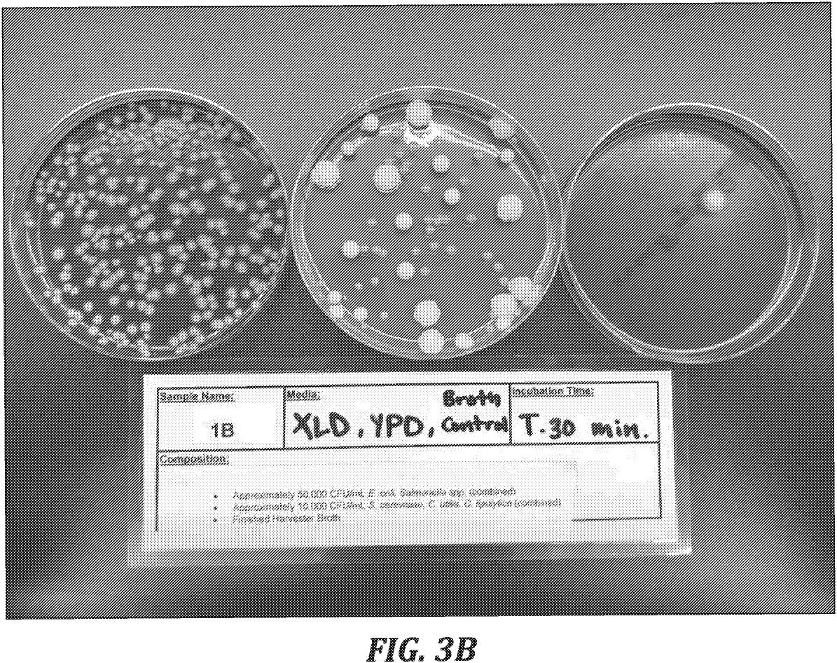

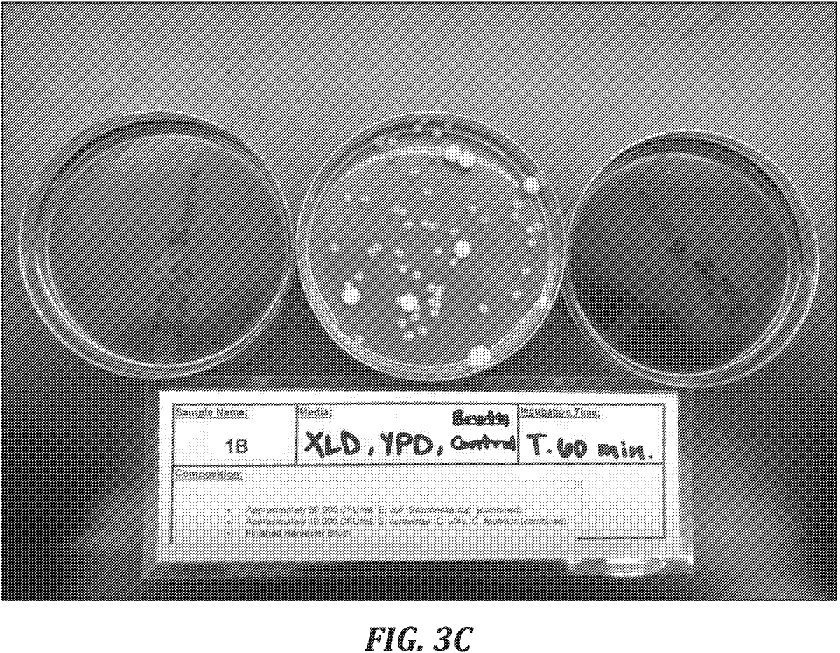

The disclosure encompasses processes that incorporate the above method embodiments to prevent putrefaction and/or increase safety of process organic biomass products, such as food, agricultural, or domestic yard and garden waste products. These processes can have several applications, such as production of nutritive-rich, safe organic fertilizer product and animal feed. FIG. 1 provides a representative schematic for a general method of producing a fertilizer product encompassed by this disclosure.

To illustrate, in one embodiment, the method is for inhibiting putrefaction in biomass and comprises:
processing a biomass to produce a substantially homogenized liquid slurry;
contacting the substantially homogenized liquid slurry with an effective amount of live non-pathogenic yeast;
agitating the substantially homogenized liquid slurry continuously to distribute the yeast within the substantially homogenized liquid slurry in aerobic conditions to provide a yeast-stabilized biomass slurry;
filtering the yeast-stabilized biomass slurry to remove macroparticles and produce a yeast-stabilized biomass slurry filtrate; and
aerating the yeast-stabilized biomass slurry filtrate.

As described above, the step of processing comprises wetting the biomass with water. In some embodiments, the water used to wet the biomass can have an elevated temperature, such as about 90° F. to about 150° F., such as 90° F., 100° F., 110° F., 120° F., 130° F., 140° F., 150° F., or within 5° F. of any of the indicated temperatures. The processing step can also include steps of crushing or grinding the biomass to provide the substantially homogenized liquid slurry. In some embodiments, the remaining solid biomass component of the substantially homogenized liquid slurry has at least 75% of particles having a diameter less than 5 mm, less than 2 mm, or less than 1 mm. In some embodiments, the method further comprises re-homogenizing and re-filtering the yeast-stabilized biomass slurry filtrate one or more times prior to the aerating step.

The yeast-stabilized biomass slurry filtrate can be maintained in its state for a prolonged period of time, for example during prolonged storage or transportation to, e.g., a centralized processing center. Additional hurdles can be applied to the yeast-stabilized biomass slurry filtrate. This can occur in the same location, either concurrently or sequentially. Additionally, the yeast-stabilized biomass slurry filtrate can be transported to a second location (e.g., a production facility) wherein additional live nonpathogenic yeast and/or one or more additional hurdles can be applied during further processing.

In some embodiments, the method further comprises contacting the yeast-stabilized biomass slurry filtrate with additional live non-pathogenic yeast, micro-nutrients comprising yeast lysate residue, and/or plant-based macronutrients. Typically, aerobic conditions are maintained with the addition of these additional components, such as by continued agitating. This supplemented yeast-stabilized biomass slurry filtrate can be further processed, including imposition of one or more of the hurdle conditions as described above (e.g., restricted pH, temperature, pressure, moisture (water activity), salt content, electrical conductivity, and redox potential). The one or more additional hurdles can be applied independently or concurrently, for similar or different durations. Any combination of the additional hurdles can be applied. The hurdles conditions can be maintained independently or together for a period of at least about 30 minutes and up to a timescale of days. Exemplary times for the hurdle conditions include at least 30 minutes, 1 hour, 2 hours, 3 hours, 4 hours, 5 hours, 6 hours, 7 hours, 8 hours, 9 hours, 10 hours, 12 hours, 18 hours, 24 hours, 2.5 days, 3 days, 4 days or more.

The supplemented yeast-stabilized biomass slurry filtrate can be re-homogenized at a temperature of selected from 70° F. to 120° F. (e.g., 80° F. to 100° F.) for at least 6 hours, followed by filtering the heated slurry one or more times to produce a refined slurry filtrate. The refined slurry filtrate can be incorporated into, e.g., a finished fertilizer product.

The following is a step by step description of an exemplary methodology encompassed by the disclosure. The main substrate ingredient in the process is preconsumer food scraps collected from grocery stores and can generally include produce, red meat, seafood, poultry, bakery and store-prepared deli foods. Following the collection of food scraps, generally following the flow diagram illustrated in FIG. 1, the process steps are as follows:
1. Initial biomass substrate (e.g., food scraps) are crushed and nearly instantaneously comminuted inside the Harvester device.
2. The receiving and grinding compartments of the Harvester are washed with water (optionally heated, e.g., to 140° F.) resulting in wetting of food scraps and cleaning of the hopper.
3. The comminuted material, typically a substantially homogenized liquid slurry, is transmitted into a receiving tank located on-site.
4. Periodic food and water additions are made throughout the day, with the volume of both ingredients varying by the amount of scrap material generated at the location and receiving tank capacity.
5. Harvester biology tanks are regularly monitored to contribute yeast (described above) and collect samples of the resulting yeast-stabilized liquid slurry for quality control purposes.
6. In the Quality Control laboratory, the yeast-stabilized liquid slurry is regularly evaluated as to pH, electrical conductivity, count of total micro-organisms, count of “seeded organisms” and count of coliform-like organisms.
7. The yeast-stabilized liquid slurry levels in the tanks are remotely monitored, and tanks are emptied when full using food-grade hoses and pumps
8. Yeast-stabilized liquid slurry is always maintained under aerobic conditions with constant mixing.
9. Collected yeast-stabilized liquid slurry is transferred using collection hoses and couplings to a polyethylene slurry receiving tank at the local processing facility.
10. Immediately upon arrival at the processing facility, the material is mechanically filtered to remove large fibrous food scraps, potential contaminants, or material that has not been sufficiently broken down. Excluded organic material is re-ground and re-processed.
11. The yeast-stabilized liquid slurry filtrate is homogenized, assisting in both further reducing particle size and releasing additional nutrients into the yeast-stabilized liquid slurry filtrate.
12. After further homogenization, the material is filtered one or more additional times and then held under aeration indefinitely until used for the creation of fertilizer product. The filtrate can be transferred to a bioprocessing tank and combined with additional components such as potassium sulfate, citric acid, and/or additional live nonpathogenic yeast (e.g., introduced as inoculum, produced as described above) as deemed necessary. Additionally, micro-supplement and macro-supplement, as described above, can be added.
13. Inoculum growth is encouraged by the slow addition of macro-supplements. Typically, over a 48 to 72-hour period the inoculated yeast species predominate and other unwanted, gratuitous flora numbers rapidly fall off. The in-process fertilizer is very stable and is maintained under aerobic conditions at 30° C. until ready for further processing.
14. After 48-72 hours, in-process fertilizer in the bioprocessing tank is transferred to a mixing tank where macro-supplement is added until the material reaches its desired guaranteed analysis.
15. The material is heated to 30° C. and the active culture is held for 48-72 hours before further processing.
16. After 48-72 hours the material is homogenized to further reduce particle size and destroy microorganisms.
17. The yeast-stabilized liquid slurry filtrate is processed sequentially through additional mechanical filtration steps and stored.
18. The product is stored under quarantine until optional QC testing is complete.
19. The product is analyzed with respect to nutrient content, metal concentrations, and levels of potential pathogens such as Salmonella species and toxigenic E. coli, and Listeria species.
20. Material that passes review is released by the laboratory manager according to standing SOP.
21. The released batch of finished refined biomass product (e.g., fertilizer) is packaged appropriately for customers (e.g., in plastic bottles, IBC totes, tanker truck, etc.).

### General Comments and Definitions

Unless specifically defined herein, all terms used herein have the same meaning as they would to one skilled in the art of the present disclosure.

For convenience, certain terms employed herein, in the specification, examples and appended claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, elements, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method step or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties

### EXAMPLES

The following examples are provided for the purpose of illustrating, not limiting, the disclosure.

### Example 1

This example describes an assay to test the ability of yeast species to reduce select pathogenic microbial growth.

### Methods

As a preliminary precaution, a sample of Synergy product was tested for the presence of radioactive contamination using an NIST-traceable scintillation detection device. The results of this testing indicated that there was no ionizing radiation detected that exceeds three standard deviations above the background atmospheric levels.

This study was undertaken to determine if a 5-log reduction could be achieved against E. coli O157:H7 (ATCC #35150), Listeria monocytogenes (ATCC #15313) and Salmonella enterica subsp. Enterica serovar Abaetetuba (ATCC #35640), when inoculated into the above product and tested for the inoculant bacteria at different intervals. Specifically, the product was inoculated separately with each of the three test organisms and then tested at different times (1 minute, 24 hours, 48 hours and 72 hours post-inoculation) to determine what, if any, log-reductions were achieved during the study.

Fresh cultures of the test organisms were prepared by streaking a single loopful from refrigerated stock culture slants onto Tryptic Soy Agar plates (TSA) and incubated for 24 hours at 35° C. A single, isolated colony from each inoculated TSA plate was transferred into Tryptic Soy Slurry (TSB) and incubated for 24 hours at 35° C. The cultures were then acid acclimated to pH 4.5 through successive, daily transfers in acidified TSB with 10% sterile Tartaric acid. Cultures were prepared in suspension and then a separate aliquot of each culture was inoculated into separate aliquots of the product to achieve a Baseline inoculum level of ~10⁶ cfu/ml.

At baseline, the inoculated products were mixed thoroughly for one minute, individual 10 gram aliquots were weighed, diluted and plated in duplicate using the FDA BAM Aerobic Plate Count Method and selective medias for each of the three pathogens: (Rapid E. coli 2 Agar for E. coli O157:H7, Modified Oxford Agar (MOX) for L. monocytogenes and Xylose-lysine-desoxycholate Agar (XLD) for S. enterica subsp. Enterica serovar Abaetetuba). A thin layer of Tryptic Soy Agar (TSA) was added to the solidified selective agars to inhibit the growth of any non-selective micro-organisms. Plates were incubated at 35° C. for 48 hours prior to enumerating. The inoculated samples were then held for an additional 24 hours, 48 hours and 72 hours stored at ambient temperature (68° F. - 72° F.) and plated accordingly. Un-inoculated samples served as controls. Test results represent an average of duplicate counts per sample tested.

### Results

The results of this study are set forth in Table 1 and indicate that the product containing Synergy product achieved a >6-log reduction against E. coli O157:H7, L. monocytogenes and S. enterica subsp. Enterica serovar Abaetetuba after 24 hours - 72 hours of ambient storage (68° F. - 72° F.). There was no recovery (<1 cfu/ml) of any of the test organisms after 24 hours, 48 hours and 72 hours at ambient storage.

Table 1

counts of bacteria in samples post inoculation

| | | | | | | | |
|---|---|---|---|---|---|---|---|
| Organism ID | Baseline Count (cfu/ml) | 24 hr. Count (cfu/ml) | Log Red. | 48 hr. Count (cfu/ml) | Log Red. | 72 hr. Count (cu/ml) | Log Red. |
| E. coli O157:H7 | 8.10E+06 | <1 | >6.91 | <1 | >6.91 | <1 | >6.91 |
| Saline Control | 9.00E+06 | 1.80E+07 | N/A | 1.30E+07 | N/A | 1.70E+07 | N/A |
| L. monocytogenes | 3.20E+06 | <1 | >6.51 | <1 | >6.51 | <1 | >6.51 |
| Saline Control | 3.70E+06 | 2.60E+06 | N/A | 2.90E+06 | N/A | 4.30E+06 | N/A |
| Salmonella Aebaet. | 5.10E+06 | <1 | >6.71 | <1 | >6.71 | <1 | >6.71 |
| Saline Control | 9.30E+05 | 4.40E+06 | N/A | 4.70E+06 | N/A | 2.40E+07 | N/A |
| Uninoculated Control | 2.40E+02 | 2.40E+03 | N/A | 2.90E+03 | N/A | 2.20E+03 | N/A |

### Conclusion

Based on these results, the product containing Synergy product formula was effective in achieving a >6-log reduction against all three test organisms after 24 hours at ambient storage.

### Example 2

This example describes an additional assay to test the ability of yeast species to reduce select pathogenic microbial growth.

### Methods

As a preliminary precaution, a sample of WISErg 3-2-2 product was tested for the presence of radioactive contamination using an NIST-traceable scintillation detection device. The results of this testing indicated that there was no ionizing radiation detected that exceeds three standard deviations above the background atmospheric levels.

This study was undertaken to determine if a 5-log reduction could be achieved against E. coli O157:H7 (ATCC #35150), Listeria monocytogenes (ATCC #15313) and Salmonella enterica subsp. Enterica serovar Abaetetuba (ATCC #35640), when inoculated into the above product and tested at different time intervals. Specifically, the product was inoculated separately with each of the three test organisms and then tested at different exposure times (1 minute, 24 hours, 48 hours and 72 hours post-inoculation) to determine what, if any, log-reductions were achieved during the study.

Fresh cultures of the test organisms were prepared by streaking a single loopful from refrigerated stock culture slants onto Tryptic Soy Agar plates (TSA) and incubated for 24 hours at 35° C. A single, isolated colony from each inoculated TSA plate was transferred into Tryptic Soy Slurry (TSB) and incubated for 24 hours at 35° C. The cultures were then acid acclimated to pH 5.0 through successive, daily transfers in acidified TSB with 6 N HCl. Cultures were prepared in suspension and then a separate aliquot of each culture was inoculated into separate aliquots of the product to achieve a Baseline inoculum level of ~10⁶ - 10⁷ cfu/ml.

At caseline, the inoculated products were mixed thoroughly for one minute, individual 10 gram aliquots were weighed, diluted and plated in duplicate using the FDA BAM Aerobic Plate Count Method and selective medias for each of the three pathogens: (Rapid E. coli 2 Agar for E. coli O157:H7, Modified Oxford Agar (MOX) for L. monocytogenes and Xylose-lysine-desoxycholate Agar (XLD) for S. enterica subsp. Enterica serovar Abaetetuba). A thin layer of Tryptic Soy Agar (TSA) was added to the solidified selective agars to inhibit the growth of any non-selective micro-organisms. Plates were incubated at 35° C. for 48 hours prior to enumerating. The inoculated samples were then held for an additional 24 hours, 48 hours and 72 hours stored at ambient temperature (68° F. - 72° F.) and plated accordingly. Un-inoculated samples served as controls. Test results represent an average of duplicate counts per sample tested.

### Results

The results of this study are set forth in Table 2 and indicate that the WISErg 3-2-2 product achieved a >6-log reduction against E. coli O157:H7, Listeria monocytogenes and Salmonella Abaetetuba after 24 hours - 72 hours of ambient storage (68° F. - 72° F.). There was no recovery (<1 cfu/ml) of any of the test organisms after 24 hours, 48 hours and 72 hours at ambient storage.

Table 2

counts of bacteria in samples post inoculation

| | | | | | | | |
|---|---|---|---|---|---|---|---|
| Organism ID | Baseline Count (cfu/ml) | 24 hr. Count (cfu/ml) | Log Red. | 48 hr. Count (cfu/ml) | Log Red. | 72 hr. Count (cu/ml) | Log Red. |
| E. coli O157:H7 | 4.50E+06 | <1 | >6.65 | <1 | >6.65 | <1 | >6.65 |
| Saline Control | 1.50E+07 | 1.80E+06 | NA | 3.10E+06 | NA | 2.70E+06 | NA |
| L. monocytogenes | 8.50E+06 | 2.30E+02 | 4.10 | <1 | >6.46 | <1 | >6.46 |
| Saline Control | 1.70E+06 | 2.60E+06 | NA | 2.90E+06 | NA | 4.30E+06 | NA |
| Salmonella Aebaet. | 9.90E+05 | <1 | >6.00 | <1 | >6.00 | <1 | >6.00 |
| Saline Control | 9.30E+05 | 4.40E+06 | NA | 4.70E+06 | NA | 2.40E+07 | NA |
| Uninoculated Control | 3.60E+02 | 5.10E+02 | NA | 3.10E+03 | NA | 5.10E+03 | NA |

### Conclusion

Based on these results, the product containing WISErg 3-2-2 product formula was effective in achieving a >6-log reduction against all three test organisms after 24 hours at ambient storage.

### Example 3

This example describes an assay to test the ability of biopreservative yeast species to reduce pathogenic microbial growth characteristic in a liquid biomass slurry (i.e., liquefied food scraps).

### Introduction

This study was directed at evaluating the effect of biopreservative organisms on pathogen reduction in input biomass slurry, which is processed from food scraps product.

### Materials

250-mL Erlenmeyer flasks
biomass slurry sampled
Lab isolates of E. coli, Salmonella, S. cerevisiae, C. utilis, C. lipolytica
Sterile 50% YPD Slurry
Sterile 1% PBS Shake incubator
Water-jacketed incubator Sterile YPD and XLD plates
Sterile pipet tips
Sterile glass plating beads

### Method

To obtain substantially homogenized liquid biomass slurry, food scraps product was sequentially wetted with 140° F. water, crushed, and comminuted inside a receiving and grinding compartments of a Harvester apparatus.

125 mL suspensions of S. cerevisiae, C. utilis and C. lipolytica were prepared from lab isolates and sterile YPD slurry, following Inoculum Preparation SOP. 100 mL of each yeast suspension were mixed together to create the combined yeast suspension.

1 Mcfarland standard equivalent solutions of E. coli and Salmonella were prepared from lab isolates and sterile 1% PBS. Equal volumes of E. coli and Salmonella solutions were added together to create the combined pathogen suspension.

Substantially homogenized liquid biomass slurry was aliquoted into 250-mL Erlenmeyer flasks and combined with the yeast and pathogen solutions as outlined below in Tables 3-6.

Table 3

Combined yeast and pathogen solutions for experimental Group 1

| | |
|---|---|
| A: 50,000 CFL/mL E. coli 10,000 CFU/mL S. cerevisiae | B: 50,000 CFL/mL E. coli 10,000 CFU/mL combined yeast |
| 40 uL E. coli suspension | 40 uL E. coli suspension |
| 1.25 mL S. cerevisiae solution | 1.25 mL S. cerevisiae solution |
| QS to 250 mL with homogenized liquid biomass slurry | QS to 250 mL with homogenized liquid biomass slurry |

Table 4

Combined yeast and pathogen solutions for experimental Group 2

| | |
|---|---|
| A: 50,000 CFL/mL Salmonella 1,000 CFU/mL S. cerevisiae | B: 50,000 CFL/mL Salmonella 100,000 CFU/mL combined yeast |
| 40 uL Salmonella suspension | 40 uL Salmonella suspension |
| 1.25 mL S. cerevisiae solution | 1.25 mL S. cerevisiae solution |
| QS to 250 mL with homogenized liquid biomass slurry | QS to 250 mL with homogenized liquid biomass slurry |

Table 5

Combined yeast and pathogen solutions for experimental Group 3

| | |
|---|---|
| A: 50,000 CFL/mL combined pathogens 10,000 CFU/mL S. cerevisiae | B: 50,000 CFL/mL combined pathogens 10,000 CFU/mL combined yeast |
| 40 uL combined pathogen suspension | 40 uL combined pathogen suspension |
| 1.25 mL S. cerevisiae solution | 1.25 mL S. cerevisiae solution |
| QS to 250 mL with homogenized liquid biomass slurry | QS to 250 mL with homogenized liquid biomass slurry |

Table 6

Combined yeast and pathogen solutions for slurry control

| | | |
|---|---|---|
| Group 1 50,000 CFL/mL E. coli | Group 2 50,000 CFL/mL Salmonella | Group 3 50,000 CFL/mL combined pathogens |
| 40 uL combined pathogen suspension | 40 uL combined pathogen suspension | 40 uL combined pathogen suspension |
| QS to 250 mL with homogenized liquid biomass slurry | QS to 250 mL with homogenized liquid biomass slurry | QS to 250 mL with homogenized liquid biomass slurry |

In addition to slurry controls outlined above, 1 McFarland (Saline) solutions of E. coli, Salmonella spp. and combined pathogens were maintained at room temperature for the duration of the experiment.

All experimental and slurry control solutions were placed in the rotary incubator at 30° C. and 200 RPM to incubate. Samples were pulled from each experimental, slurry and saline control solutions at time 0, 3 hours, 6 hours, 9 hours and 12 hours of incubation. Experimental and slurry control samples were plated on XLD at 10⁻² dilution and YPD at 10⁻⁴ dilution. Saline control samples were plated on XLD only at 10⁻¹ dilution. The XLD plates were incubated for 24 hours at 37° C. and YPD plates were incubated for 48 hours at 30° C. Following incubation all plates were evaluated for growth. Pathogen counts were recorded from XLD plates and yeast counts were recorded from YPD plates.

### Results

Results of all experimental and control groups are outlined in Tables 7-9 below.

Table 7

Bacterial counts for experimental Group 1

| | A | | B | | Slurry Control | | Saline Control |
|---|---|---|---|---|---|---|---|
| Incubation Time | XLD (CFU/mL) | YPD (CFU/mL) | XLD (CFU/mL) | YPD (CFU/mL) | XLD (CFU/mL) | YPD (CFU/mL) | XLD (CFU/mL) |
| 0 Hours | >3e4 | >3e6 | >3e4 | >3e6 | >3e4 | >3e6 | >3e3 |
| 3 Hours | <1e2 | >3e6 | <1e2 | >3e6 | <1e2 | >3e6 | >3e3 |
| 6 Hours | <1e2 | >3e6 | <1e2 | >3e6 | <1e2 | >3e6 | >3e3 |
| 9 Hours | <1e2 | >3e6 | <1e2 | 1.70E+02 | <1e2 | 1.50E+06 | >3e3 |
| 12 Hours | <1e2 | 1.80E+06 | <1e2 | 5.50E+05 | <1e2 | 1.20E+06 | >3e3 |

Table 8

Bacterial counts for experimental Group 2

| | A | | B | | Slurry Control | | Saline Control |
|---|---|---|---|---|---|---|---|
| Incubation Time | XLD (CFU/mL) | YPD (CFU/mL) | XLD (CFU/mL) | YPD (CFU/mL) | XLD (CFU/mL) | YPD (CFU/mL) | XLD (CFU/mL) |
| 0 Hours | 3.90E+03 | >3e6 | 1.20E+03 | >3e6 | 1.00E+03 | >3e6 | >3e3 |
| 3 Hours | <1e2 | >3e6 | <1e2 | >3e6 | <1e2 | >3e6 | >3e3 |
| 6 Hours | <1e2 | >3e6 | <1e2 | >3e6 | <1e2 | >3e6 | >3e3 |
| 9 Hours | <1e2 | >3e6 | <1e2 | 1.60E+06 | <1e2 | 1.70E+06 | >3e3 |
| 12 Hours | <1e2 | 1.80E+06 | <1e2 | 1.40E+06 | <1e2 | 7.50E+05 | >3e3 |

Table 9

Bacterial counts for experimental Group 3

| | A | | B | | Slurry Control | | Saline Control |
|---|---|---|---|---|---|---|---|
| Incubation Time | XLD (CFU/mL) | YPD (CFU/mL) | XLD (CFU/mL) | YPD (CFU/mL) | XLD (CFU/mL) | YPD (CFU/mL) | XLD (CFU/mL) |
| 0 Hours | 1.80E+04 | >3e6 | 1.50E+04 | >3e6 | >3e4 | >3e6 | >3e3 |
| 3 Hours | <1e2 | >3e6 | <1e2 | >3e6 | <1e2 | >3e6 | >3e3 |
| 6 Hours | <1e2 | >3e6 | <1e2 | >3e6 | <1e2 | >3e6 | >3e3 |
| 9 Hours | <1e2 | >3e6 | <1e2 | 2.00E+06 | <1e2 | 1.20E+06 | >3e3 |
| 12 Hours | <1e2 | 1.40E+06 | <1e2 | 9.20E+05 | <1e2 | 8.20E+05 | >3e3 |

### Conclusions

Pathogens in the Saline controls remained viable for the duration of the experiment. In contrast, viable pathogens were eliminated from all experimental and slurry control groups within 3 hours of experimental initiation establishing that these procedures are effective to kill and eliminate potentially harmful pathogens from the processed biomass.

### Example 4

This example describes an additional assay to test the ability of biopreservative yeast species to reduce pathogenic microbial growth characteristic in substantially homogenized liquid biomass slurry (i.e., liquefied organic waste).

### Introduction

This study was directed at evaluating the effect of biopreservative organisms on pathogen reduction in input homogenized liquid biomass slurry, which is processed from food scraps product. This study aims to further isolate the effect biopreservative organisms have on pathogen concentration by eliminating the presence of viable background yeast found in homogenized liquid biomass slurry, as was used in Example 3. Additionally, samples will be evaluated at shorter intervals compared to Example 3 in an effort to observe a more gradual decline in pathogen concentrations.

### Materials

250-mL Erlenmeyer flasks
Sterilized homogenized liquid biomass slurry sampled from the BH2 tank Lab isolates of E. coli, Salmonella, S. cerevisiae, C. utilis, C. lipolytica
Sterile 50% YPD Slurry
Sterile 1% PBS Shake incubator
Water-jacketed incubator Sterile YPD and XLD plates
Sterile pipet tips
Sterile glass plating beads

### Method

Substantially homogenized liquid biomass slurry was obtained as described in Example 3, above.

125 mL suspensions of S. cerevisiae, C. utilis and C. lipolytica were prepared from lab isolates and sterile YPD slurry, following Inoculum Preparation SOP. 100 mL of each yeast suspension were mixed together to create the combined yeast suspension.

1 Mcfarland standard equivalent solutions of E. coli and Salmonella were prepared from lab isolates and sterile 1% PBS. Equal volumes of E. coli and Salmonella solutions were mixed together to create the combined pathogen suspension.

Sterile liquid biomass slurry from the BH2 tank was aliquoted into 250-mL Erlenmeyer flasks and combined with yeast and pathogen solutions as outlined below in Tables 10 and 11.

Table 10

Combined yeast and pathogen solutions for experimental Group 1

| | |
|---|---|
| A: 50,000 CFL/mL combined pathogens 10,000 CFU/mL S. cerevisiae | B: 50,000 CFL/mL combined pathogens 10,000 CFU/mL combined yeast |
| 40 uL E. coli suspension | 40 uL E. coli suspension |
| 1.25 mL S. cerevisiae solution | 1.25 mL S. cerevisiae solution |
| QS to 250 mL with homogenized liquid biomass slurry | QS to 250 mL with homogenized liquid biomass slurry |

Table 11

Combined yeast and pathogen solutions for slurry control
Group 1 50,000 CFL/mL combined pathogens
40 uL combined pathogen suspension
QS to 250 mL with homogenized liquid biomass slurry

### Results

Results of all experimental and control groups are outlined in Table 12.

Table 12

Bacterial counts for experimental Group 1

| | A | | B | | Slurry Control | | Saline Control |
|---|---|---|---|---|---|---|---|
| Incubatio n Time (minutes) | XLD (CFU/mL) | YPD (CFU/mL) | XLD (CFU/mL) | YPD (CFU/mL) | XLD (CFU/mL) | YPD (CFU/mL) | XLD (CFU/mL) |
| 0 | 8.00E+02 | 2.20E+05 | 1.40E+03 | 1.00E+05 | 2700 | <1e4 | >3e3 |
| 30 | 200 | 2.5E+05 | <100 | 1.20E+05 | 100 | <1e4 | >3e3 |
| 60 | <100 | 3.6E+05 | <100 | 1.00E+05 | <100 | <1e4 | >3e3 |
| 90 | <100 | 3.1E+05 | <100 | 2.40E+05 | <100 | <1e4 | >3e3 |
| 120 | <100 | 2.1E+05 | <100 | 3.80E+05 | <100 | <1e4 | >3e3 |
| 150 | <100 | 3.0E+05 | <100 | 1.50E+05 | <100 | <1e4 | >3e3 |
| 180 | <100 | 3.8E+05 | <100 | 9.00E+04 | <100 | <1e4 | >3e3 |

### Conclusions

Pathogens in the saline controls remained viable for the duration of the experiment. Viable pathogens were eliminated from all experimental and slurry control groups within 60 minutes of experimental initiation. This demonstrates that culture of select yeast species in the organic slurry inhibits growth and even eliminates the detectable presence of pathogenic microorganisms that can lead to putrefaction of the substrate.

While illustrative embodiments have been illustrated and described, it will be appreciated that various changes can be made therein without departing from the spirit and scope of the invention.

1. A method of inhibiting pathogenic microbial growth in a biomass, comprising:
contacting the biomass with an effective amount of live non-pathogenic yeast;
agitating the biomass to distribute the yeast within the biomass to provide a yeast-stabilized biomass slurry; and
maintaining aerobic conditions in the slurry to permit yeast to grow aerobically.

2. The method of claim 1, further comprising processing the biomass to produce a substantially homogenized liquid slurry prior to contacting with the effective amount of live non-pathogenic yeast.

3. The method of claim 2, wherein the processing comprises crushing or grinding the biomass to provide the substantially homogenized liquid slurry with at least 80% of biomass being particles with a diameter less than 2 mm.

4. The method of claim 1, wherein the biomass comprises one of food, food scraps, waste products, agricultural waste products, domestic yard waste products, and combinations thereof.

5. The method of claim 1, wherein the live non-pathogenic yeast comprises yeast selected from Saccharomyces and Candida, and combinations thereof.

6. The method of claim 5, wherein the live non-pathogenic yeast comprises a yeast species selected from Saccharomyces cerevisiae, Candida utilis, and Candida lipolytica, and combinations thereof.

7. The method of claim 1, wherein the live non-pathogenic yeast contacted with the biomass is metabolically active.

8. The method of claim 1, wherein the effective amount of live non-pathogenic yeast is at least 1E⁴ CFU/mL of slurry.

9. The method of claim 1, wherein the effective amount of live non-pathogenic yeast is added to the biomass continuously while agitating the biomass to create the yeast-stabilized biomass slurry.

10. The method of claim 1, wherein the effective amount of live non-pathogenic yeast is contacted in a plurality of discrete doses over time sufficient to maintain a population of live non-pathogenic yeast of at least 1E⁴ CFU/mL of slurry.

11. The method of claim 1, further comprising adding a micro-nutrient comprising yeast lysate residue to the yeast-stabilized biomass slurry.

12. The method of claim 1, further comprising adding a macronutrient to the yeast-stabilized biomass slurry.

13. The method of claim 1, further comprising maintaining a temperature in the yeast-stabilized biomass slurry selected from 50° F. to 120° F. for at least 30 minutes.

14. The method of claim 14, wherein the temperature is elevated to at least 100° F. for at least 30 minutes.

15. The method of claim 1, further comprising maintaining at least a portion of the yeast-stabilized biomass slurry under a pressure of at least 2 bars for at least 30 seconds.

16. The method of claim 15, wherein with mixing the elevated pressure is applied to each portion of the yeast-stabilized biomass slurry for at least 30 seconds.

17. The method of claim 15, wherein the pressure is maintained within the yeast-stabilized biomass slurry at a pressure selected from 5 bars to 16 bars for at least 30 minutes.

18. The method of claim 1, further comprising maintaining the yeast-stabilized biomass slurry at a pH less than 5 for at least 30 minutes.

19. The method of claim 18, wherein the yeast-stabilized biomass slurry is maintained at a pH of 4.2±0.5 for at least 30 minutes.

20. The method of claim 18, wherein maintaining the pH comprises adding one or more acids.

21. The method of claim 1, further comprising maintaining the yeast-stabilized biomass slurry at a water activity less than 0.97 A_{W} for at least 30 minutes.

22. The method of claim 21, wherein the yeast-stabilized biomass slurry is maintained at a water activity less than 0.95 A_{W}, 90 A_{W}, or 85 A_{W} for at least 30 minutes.

23. The method of claim 1, further comprising maintaining the yeast-stabilized biomass slurry at an electrical conductivity (EC) of 20.0±5 mS/cm for at least 30 minutes.

24. The method of claim 1, further comprising maintaining the yeast-stabilized biomass slurry at a redox potential (Eh) selected from 0 mV to -200 mV for at least 30 minutes.

25. The method of claim 1, wherein maintaining aerobic conditions comprises agitating the yeast-stabilized biomass slurry continuously or periodically, and ventilating or aerating the yeast-stabilized biomass slurry with gas comprising oxygen.

26. The method of claim 1, wherein the pathogenic microbial growth is reduced compared to pathogenic microbial growth in equivalent biomass that is not contacted with the live non-pathogenic yeast.

27. The method of claim 1, wherein the pathogenic microbes are selected from the genera Lactobacillus, Enterobacter, Salmonella, and Escherichia.

28. A method of inhibiting putrefaction in biomass, comprising:
processing a biomass to produce a substantially homogenized liquid slurry;
contacting the substantially homogenized liquid slurry with an effective amount of live non-pathogenic yeast;
agitating the substantially homogenized liquid slurry continuously to distribute the yeast within the substantially homogenized liquid slurry in aerobic conditions to provide a yeast-stabilized biomass slurry;
filtering the yeast-stabilized biomass slurry to remove macroparticles to produce a yeast-stabilized biomass slurry filtrate; and
aerating the yeast-stabilized biomass slurry filtrate.

29. The method of claim 28, wherein the processing comprises wetting the biomass with water.

30. The method of claim 28, wherein the processing comprises crushing or grinding the biomass to provide substantially homogenized liquid slurry with at least 80% of biomass particles having a diameter less than 2 mm.

31. The method of claim 28, further comprising re-homogenizing and re-filtering the yeast-stabilized biomass slurry filtrate one or more times prior to the aerating step.

32. The method of claim 28, further comprising: contacting the yeast-stabilized biomass slurry filtrate with the following:
live non-pathogenic yeast;
micro-nutrients comprising yeast lysate residue; and
macronutrients; and
maintaining aerobic conditions.

33. The method of claim 32, further comprising maintaining a temperature of the yeast-stabilized biomass slurry selected from 50° F. to 120° F. for at least 30 minutes.

34. The method of claim 32, further comprising maintaining a temperature of the yeast-stabilized biomass slurry selected from 75° F. to 90° F. for at least 30 minutes.

35. The method of claim 32, further comprising elevating the temperature in the yeast-stabilized biomass slurry to at least 100° F. for at least 30 minutes.

36. The method of claim 32, further comprising maintaining at least a portion of the yeast-stabilized biomass slurry under a pressure of at least 2 bars for at least 30 seconds.

37. The method of claim 36, wherein with mixing the elevated pressure is applied to each portion of the yeast-stabilized biomass slurry for at least 30 seconds.

38. The method of claim 36, wherein the pressure is maintained within the yeast-stabilized biomass slurry at a pressure selected from 5 bars to 16 bars for at least 30 minutes.

39. The method of claim 32, further comprising maintaining the yeast-stabilized biomass slurry at a pH of less than 5 for at least 30 minutes.

40. The method of claim 39, wherein the pH in the yeast-stabilized biomass slurry is maintained at 4.2±0.5 for at least 30 minutes.

41. The method of claim 39, wherein the pH is maintained by addition of one or more acids.

42. The method of claim 32, further comprising maintaining the yeast-stabilized biomass slurry at a water activity less than 0.97 A_{W} for at least 30 minutes.

43. The method of claim 42, wherein the yeast-stabilized biomass slurry is maintained at water activity less than 0.95 A_{W}, 90 A_{W}, or 85 A_{W} for at least 30 minutes.

44. The method of claim 1, further comprising maintaining the yeast-stabilized biomass slurry at an electrical conductivity (EC) of 20.0±5 mS/cm for at least 30 minutes.

45. The method of claim 1, further comprising maintaining the yeast-stabilized biomass slurry at a redox potential (Eh) selected from 0 mV to -200 mV for at least 30 minutes.

46. The method of claim 32, further comprising re-homogenizing the yeast-stabilized biomass slurry at a temperature of 75° F. to 90° F. for at least 6 hours, followed by filtering the heated slurry one or more times to produce a refined slurry filtrate.
